SEQUENCE LISTING (1 sequence)

SEQ ID NO: 1 - protein, 12 aa, Artificial Sequence
  Synthetic construct
MRGSXXXXXXGS

(19) United States
(12) Patent Application Publication
(10) Pub. No.: US 2009/0098157 A1
(43) Pub. Date: Apr. 16, 2009

(54) Helicobacter Pylori Vaccination

(75) Inventor: Giuseppe Del Giudice, Siena (IT)

Correspondence Address: Melodi Dice, Novartis Vaccines and Diagnostics, Ins., 4560 Horton Street, Emeryville, CA, 94608-2916, US

(73) Assignee: Novartis Vaccines and Diagnostics s.r.I., Siena SI (IT)

(21) Appl. No.: 12/233,980

(22) Filed: Sep. 19, 2008

Related U.S. Application Data
(62) Division of application No. 10/487,962, filed on Mar. 14, 2005, now abandoned.

(30) Foreign Application Priority Data
Aug. 31, 2001 (GB) GB0121208.3
Oct. 25, 2001 (GB) GB0125665.0
Mar. 4, 2002 (GB) GB0205018.5

(51) Int. Cl. A61K 39/29; A61K 39/02; C12Q 1/58; A61K 39/09

(52) U.S. Cl. 424/201.1; 424/236.1; 424/203.1; 435/12

(57) ABSTRACT

A sterile immunogenic preparation of three purified H. pylori antigens (CagA, VacA and NAP) adjuvanted with alum in an isotonic buffer solution for intramuscular injection. The antigens may be administered in conjunction with antibiotics and/or antisecretories. Urease breath testing, stool antigen testing, and/or immunological analysis may be used as correlate(s) of protection against H. pylori infection. Urea may be used to improve VacA solubility.

All documents cited herein are incorporated by reference in their entirety.

### TECHNICAL FIELD

This invention is in the field of vaccines against Helicobacter pylori.

### BACKGROUND ART

Helicobacter pylori (HP) is a Gram-negative spiral bacterium which infects the human stomach. It is believed that over 50% of the world's population harbour the bacterium.

Because of the high prevalence of HP infection and its acquisition in childhood, global eradication of disease caused by HP can only be achieved by widespread vaccination. Prevention of HP infection in a given individual would be expected to decrease the likelihood of that individual subsequently developing gastroduodenal ulcer disease or gastric cancer.

Various antigenic proteins have been identified in HP [e.g. references 1 to 5], including urease, VacA, CagA, NAP, flagella proteins, adhesins etc. and many of these have been proposed for use in vaccines. Two complete HP genome sequences are also available [6,7].

The feasibility of prophylactic vaccination against HP infection has been demonstrated in both small and large animal models. A mouse model of infection [8] was developed based upon the ability to infect mice with HP strains freshly isolated from patients with peptic ulcer disease. Oral immunisation of mice with three recombinant HP antigens (VacA, CagA, and NAP), singly or in combination, together with mucosal adjuvants (e.g. enterotoxin LT from wild type E. coli or the non-toxic K63 mutant) was shown to protect against subsequent challenge with HP [9,10]. Moreover, VacA (native and recombinant form p95) protected against challenge with a type I (VacA⁺) but not a type II (VacA⁻) HP strain. Protection therefore appears to be antigen-specific. It is an object of the invention to provide a HP vaccine for clinical use in humans.

### DISCLOSURE OF THE INVENTION

The vaccine of the invention is a sterile preparation of three purified HP antigens, adjuvanted with alum, in an isotonic buffer solution for intramuscular injection. The three antigens in this formulation are CagA, VacA and NAP. Each of these is involved in infection pathogenesis and has demonstrated immunogenicity and prophylactic efficacy in preclinical testing.

The invention therefore provides a composition comprising: (a) H. pylori CagA, VacA and NAP proteins; (b) an aluminium salt adjuvant; and (c) a buffer solution.

The invention also provides a process for producing such a composition, comprising admixing H. pylori CagA, VacA and NAP proteins, an aluminium salt adjuvant, and a buffer solution. These five components may be mixed in any order; the preferred order of mixing the proteins is to add CagA to NAP, and then add VacA to the CagA/NAP mixture.

### The Proteins

CagA, VacA and NAP proteins can be produced in any suitable manner. They may be purified from HP but, more typically, they will be purified from a recombinant expression system.

Recombinant expression preferably utilises a bacterium, and most preferably utilises E. coli. The bacteria will generally contain plasmids which encode the proteins of the invention. It is preferred that the proteins are expressed separately, rather than co-expressing the proteins in the same bacterium. After purification of the separate proteins, they may then be combined during preparation of the compositions of the invention. Preferably, therefore, the proteins are expressed in different bacteria (e.g. by using plasmids in different bacteria, each plasmid directing the expression of one of the three antigens) rather than in the same bacterium.

CagA, VacA and NAP proteins are preferably each prepared in purified form prior to being combined to form the composition of the invention. The degree of purity for each antigen prior to combination is preferably ≧90% (w/w) for each antigen i.e. the amount of CagA, VacA or NAP is at least 90% by weight of the total amount of protein. More preferably, the degree of purity is at least 91% (e.g. ≧92%, ≧93%, ≧94%, ≧95%, ≧96%, ≧97%, ≧98%, ≧99%).

The proteins can, of course, be prepared by various means (e.g. native expression, recombinant expression, purification from H. pylori culture, chemical synthesis etc.) and in various forms (e.g. native, fusions etc.). They are preferably prepared in substantially pure form (i.e. substantially free from other bacterial or host cell proteins). The proteins may each be in solution or in dry form (e.g. lyophilised) prior to their combination, but it is preferred that they are in solution. The protein concentrations in the solutions are assessed and then the appropriate volume of each is used to give a desired concentration of each protein in the final mixture.

### CagA Antigen

CagA (cytotoxicity-associated antigen) is the protein that is actively injected into epithelial cells during in vivo HP infection. After tyrosine phosphorylation and binding to a host protein, CagA activates a signaling cascade, actin remodeling, IL-8 production and other responses [11]. CagA was identified as an immunodominant antigen, present in the majority of HP strains [12,13,14]. Most individuals infected with CagA⁺ strains mount an antibody response against this antigen. Furthermore, most CD4⁺ T lymphocytes infiltrating the gastric mucosa of infected individuals are specific for CagA. The theoretical mass of CagA is ˜128 kDa, with a size variability obtained via internal duplications which generates sequences already present in the antigen, without producing antigenic diversity [13]. The protein is otherwise relatively conserved in sequence variability [6,7].

Any suitable form of CagA can be used in accordance with the invention e.g. allelic and polymorphic forms [e.g. 15], variants, mutants, immunogenic fragments etc. Identifying the CagA gene in any given HP strain is straightforward, particularly in light of the available HP genomic sequences [e.g. refs. 6 & 7].

A preferred form of CagA is a 1147 residue protein having the sequence given in reference 13, but having a substitution of threonine-382 with alanine. This protein has a main protein band of about 100 kDa as shown by SDS-PAGE analysis.

### VacA Antigen

VacA (vacuolating toxin) is released in vivo from H. pylori as a high MW homo-oligomer. Each monomer consists of a 95 kDa polypeptide which undergoes proteolytic processing to produce two fragments: one (p37) containing the enzymatic activity, and the other (p58) containing the region of binding to a gastric epithelial cell receptor [9,16]. The protein assembles to form hexa- or hepta-meric “flower-like” structures with high MW. The amino acid sequence of the VacA cytotoxin is well conserved, except for a part of the p58, called mid-region or “m”, which expresses allelic variation [6,7,17].

Any suitable form of VacA can be used in accordance with the invention e.g. allelic and polymorphic forms [e.g. 15], variants, mutants, immunogenic fragments etc. Identifying the VacA gene in any given HP strain is straightforward, particularly in light of the available HP genomic sequences [e.g. refs. 6&7].

Although wild-type VacA is associated with vacuolation of the gastric mucosa, the VacA used in the compositions of the invention is preferably in a form which does not possess any vacuolating activity. This may be due, for instance, to mis-folding [18] or to partial or complete denaturation (e.g. by formaldehyde treatment [19]).

A preferred form of VacA is a 980 amino acid molecule beginning at its amino-terminus with the amino acid sequence NH₂-Met-Arg-Gly-Ser-Xaa-Xaa-Xaa-Xaa-Xaa-Xaa-Gly-Ser- and continuing with residues 34 to 1001 of the sequence from reference 16. Each of the six Xaa residues can be the same or different as the others, and each can be any amino acid (e.g. Glu, Arg, or His). This antigen has a main protein band between 95-100 kDa as shown by SDS-PAGE analysis.

### NAP Antigen

NAP (neutrophil-activating protein) is a highly conserved antigen in all strains of H. pylori [6,7,20, 21,22]. It is a virulence factor important for the HP pathogenic effects at the site of infection and a candidate antigen for vaccine development. NAP protein activates human neutrophils and monocytes, and promotes their chemotaxis. The majority of HP-infected patients produce NAP-specific antibodies, suggesting an important role of this factor in immunity. This activity is potentiated by TNF-α and IFN-γ, is inhibited by pertussis toxin (suggesting that NAP activity is exerted through a G protein), and is sensitive to wartmannin (suggesting that NAP activity is exerted through a PI3-kinase). It has been also shown that vaccination of mice with NAP antigen induces protection against HP challenge [10]. NAP is a 17 kDa monomer, rich in alpha helices (80% of the structure), that assembles to form dodecameric structures and binds up to 40 atoms of iron per monomer [23].

Any suitable form of NAP can be used in accordance with the invention e.g. allelic and polymorphic forms, variants, mutants, immunogenic fragments etc. Identifying the NAP gene in any given HP strain is straightforward, particularly in light of the available HP genomic sequences [e.g. refs. 6 & 7]. NAP is preferably included in multimeric form.

A preferred form of NAP is a 144 amino acid protein having the sequence set out in reference 20, but with substitution of lysine-8 with arginine, leucine-58 with isoleucine, and aspartic acid-80 with glutamic acid [24]. This antigen has a main protein band of approximately 15 kDa as shown by SDS-PAGE analysis.

### Alum Adjuvant

The choice of the alum adjuvant was based on the observation that infected animals and humans exhibit a prominent Th1-type immune response, whereas a Th2-type response is more frequently encountered in individuals with mild HP infection [25]. Alum is recognised to be a strong inducer of Th2-type responses, both in animals and humans. Consequently, safety and adjuvanticity must be balanced between obtaining maximum immune stimulation with minimum side effects. Aluminium salts, including aluminium hydroxides (alum), are presently the only adjuvants approved by the FDA for use in humans. Billions of doses have been administered to children and infants, and their safety has been demonstrated with extensive clinical use. Although side effects include erythema, contact hypersensitivity, subcutaneous nodules, and granulomatous inflammation, little or no systemic toxicity is generally seen [26].

The composition of the invention comprises an aluminium salt as adjuvant. Suitable aluminium salts include hydroxide, phosphate, hydroxyphosphate, oxyhydroxide, orthophosphate, sulphate etc. (e.g. see chapters 8 & 9 of ref. 27). Mixtures of different aluminium salts may also be used. The salt(s) may take any suitable form (e.g. gel, crystalline, amorphous etc.).

A preferred amount of aluminium salt is about 0.5 mg per dose.

Aluminium hydroxides are the preferred salts for use according to the invention.

CagA, VacA and NAP are preferably adsorbed to the aluminium salt.

### Formulation

The compositions of the invention may be formulated in unit dosage form. VacA, CagA and NAP are preferably present at a concentration such that a single dose administered to a patient will contain between 10 μg and 50 μg of each of the three proteins. The amount of each protein per dose may be the same or different, so the total amount of the three proteins can vary anywhere between 30 μg and 150 μg.

A preferred composition comprises 10 μg of each protein per dose (i.e. 30 μg in total). Another preferred composition comprises 25 μg of each protein per dose (i.e. 75 μg in total).

A single dose of the composition will typically have a volume of about 500 μl.

Compositions of the invention comprise a buffer solution. The composition is preferably buffered to a pH of between 6 and 8, more preferably between 6.5 and 7.5, and most preferably about 7. This will typically be achieved using a phosphate buffer, although other buffers (e.g. histidine buffer) may also be used.

Compositions of the invention may also include components which enhance protein solubility (e.g. denaturing agents, such as urea or guandinium hydrochloride). These are particularly useful for ensuring that VacA remains soluble (i.e. the amount should be sufficient to ensure that VacA remains soluble). Preferred compositions of the invention may therefore include a low level of urea e.g. between 2.9 mg/dose and 4.1 mg/dose. These concentrations are not considered to be a safety concern—urea is normally present in blood at 60-200 mg/l-, and has been administered in some clinical settings to induce hyperosmolality. Favourable safety data in rabbits using 3.75 mg/dose and 7.5 mg/dose have also been obtained. The urea may be added to the composition as a separate component; typically, however, it will be added together with VacA because it will already be present in the purified VacA composition.

The invention also provides a composition comprising VacA and urea.

Compositions of the invention may also include low levels of a preservative, such as phenoxyethanol (e.g. about 0.5%).

Compositions of the invention may include trace amounts of antibiotics, such as chloramphenicol.

Composition of the invention are preferably isotonic with respect to human tissue.

Compositions of the invention are preferably sterile. This may be achieved by any convenient means e.g. by filter sterilisation of the components prior to mixing.

The composition may comprise components in addition to those specified herein. For example, the composition may include components in addition to (a), (b) and (c), but it may consist of (or consist essentially of) components (a), (b) and (c).

### Route and Method of Administration

Once formulated, the compositions of the invention can be administered to a patient. The patients to be treated can be animals; in particular, human subjects can be treated.

The comparative immunogenicity and prophylactic efficacy of vaccination by different routes (intragastric, intramuscular, and intranasal) was examined in the Beagle model [28] using either whole cell HP lysate or a combination of CagA, VacA and NAP. Alum adjuvant was used in each case. Antigen doses ranged from 10 through 250 μg per antigen. It was found that the intramuscular route of immunisation is superior to the intragastric and intranasal routes.

It is therefore preferred that the compositions of the invention are adapted for administration by the intramuscular route. Other possible parenteral routes of administration for direct delivery of the compositions include subcutaneous injection and intravenous injection. The compositions can also be administered into a lesion, or by oral and pulmonary administration, suppositories, transdermal or transcutaneous applications [e.g. reference 29] and hyposprays.

The compositions are preferably prepared as injectables, either as liquid solutions or suspensions or, alternatively, as solid forms suitable for solution in, or suspension in, liquid vehicles prior to injection. Any substances in the composition should preferably be compatible with intramuscular injection. Administration will typically require injection using a needle e.g. a 1-½ inch (2.5-4 cm; 21-25 gauge) needle. The composition is preferably located within a syringe.

As an alternative, the composition may be administered by needle-free means [e.g. reference 30].

Dosage treatment may be a single dose schedule or a multiple dose schedule, which may include booster doses. The composition is preferably intramuscularly administered to a patient three times in a single course of treatment, optionally followed by a fourth (booster) dose. Administration is preferably to the upper arm (M. deltoideus). Where a treatment comprises more than one administration, it is convenient to alternate the left and right arms.

The composition is preferably stored in a refrigerator (e.g. between 2° C. and 8° C.) prior to administration to a patient.

### Immunogenic Compositions and Medicaments

The compositions of the invention are preferably immunogenic composition, and are more preferably vaccine compositions.

Vaccines according to the invention may either be prophylactic (i.e. to prevent infection) or therapeutic (i.e. to treat infection), but will typically be prophylactic.

The invention also provides a composition of the invention for use as a medicament. The medicament is preferably able to raise an immune response in a mammal against CagA, VacA and NAP (i.e. it is an immunogenic composition) and is more preferably a vaccine.

The invention also provides the use of a composition of the invention in the manufacture of a medicament for raising an immune response in a mammal against the CagA, VacA and NAP. The medicament is preferably a vaccine.

The invention also provides a method for raising an immune response in a mammal comprising the step of administering an effective amount of a composition of the invention. The immune response is preferably protective. The method may raise a booster response.

The mammal is preferably a human. Where the vaccine is for prophylactic use, the human is preferably a child (e.g. a toddler or infant); where the vaccine is for therapeutic use, the human is preferably an adult. A vaccine intended for children may also be administered to adults e.g. to assess safety, dosage, immunogenicity, etc.

These uses and methods are preferably for the prevention and/or treatment of a disease caused by Helicobacter pylori (e.g. chronic gastritis, duodenal and gastric ulcer disease, gastric adenocarcinoma).

### Assessing Vaccine Efficacy

To assess efficacy as an immunogenic composition or as a vaccine, compositions of the invention may be tested in animal models of H. pylori infection [e.g. see pages 530-533 of reference 1]. The presence or absence of H. pylori infection can be assessed using one or more invasive (e.g. endoscopy with biopsy, culture, urease testing) and/or non-invasive (e.g. urease breath test, stool antigen) approaches.

To assess prophylactic efficacy in a human subject, it is preferred to use one, two or all of the following non-invasive methods: the urease breath test (UBT), stool antigen shedding, and/or analysis of immune response. The presence of H. pylori antigens in stools indicates active infection, as does a positive result in UBT. The appearance of anti-H. pylori antibodies indicates that the composition of the invention has provoked an immune response. Prophylactic efficacy can therefore be assessed by continued negative results in stool antigen or UBT assays, and immunogenicity can be assessed by the development of a positive immune response (antibody or cellular) in any biological fluid. These methods are preferably used singly or in combination to give a correlate of protection, optionally in combination with invasive methods such as biopsy.

The UBT is widely used to detect and/or diagnose H. pylori infection [e.g. refs. 31 & 32]. It typically involves the measurement of labelled CO₂ following oral administration of isotopically-labelled urea. UBT has been used to monitor H. pylori eradication by antibiotic therapy, but it has not previously been used to monitor prophylactic efficacy.

The presence of H. pylori antigens in stools has also been used to monitor H. pylori therapy [e.g. ref. 33], but this test has not been used to monitor prophylactic efficacy or the efficacy of therapeutic immunisation. The test generally measures antigens using polyclonal sera, so is not specific to any particular H. pylori antigens. It is also possible, however, to measure particular antigens (e.g. CagA, VacA) which are H. pylori-specific.

Immunological testing has been widely used for monitoring both infection and vaccine immunogenicity. Serological testing is typical. For the compositions of the invention, the presence of antibodies against the antigens in the composition (i.e. against CagA, VacA and/or NAP) indicates that it has successfully provoked an immune response. The antibodies may be of any type (e.g. IgA, IgG, IgM etc.), and may be measured in any biological fluid, but it is preferred to test IgG in serum. The test is preferably semi-quantitative or quantitative, with quantitative ELISA being the most preferred way of assessing serological response.

The same tests can be used to monitor the therapeutic efficacy of a composition of the invention, although efficacy will be determined differently. For example, rather than monitoring for the failure of a positive UBT response to appear, the loss of a positive response will be monitored.

### Compositions of the Invention

The invention provides a composition comprising: (a) H. pylori CagA, VacA and NAP proteins; (b) an aluminium salt adjuvant; and (c) a buffer solution, wherein CagA, VacA and NAP are each present at a concentration of between 20 μg/ml and 100 μg/ml.

The invention also provides a composition comprising: (a) H. pylori CagA, VacA and NAP proteins; (b) an aluminium salt adjuvant; (c) a buffer solution; and (d) urea.

The invention also provides a composition in unit dosage form comprising (a) H. pylori CagA, VacA and NAP proteins; (b) an aluminium salt adjuvant; and (c) a buffer solution, wherein CagA, VacA and NAP are each present at a concentration of between 10 μg/dose and 50 μg/dose.

The invention also provides a kit comprising a composition of the invention and an antisecretory agent and/or an antibiotic effective against Helicobacter pylori.

Two preferred compositions of the invention consist essentially of the following components per dose (e.g. per 0.5 ml dose) and have a pH in the range 7.0 to 8.0:

| | Amount per final dose | | | |
|---|---|---|---|---|
| | First | | Second | |
| Component | composition | | composition | |
| Aluminium hydroxide adjuvant | 0.5 | mg | 0.5 | mg |
| NAP | 10 | μg | 25 | μg |
| CagA | 10 | μg | 25 | μg |
| VacA | 10 | μg | 25 | μg |
| Sodium phosphate | 10 | mM | 10 | mM |
| (NaH₂PO₄•H₂O) | | | | |
| Sodium chloride (NaCl) | 2.13-2.77 | mg | 2.13-2.77 | mg |
| Urea | 2.9-4.1 | mg | 2.9-4.1 | mg |
| H₂O | Up to 0.5 | mL | Up to 0.5 | mL |

Further components of the composition

The composition of the invention will typically, in addition to the components mentioned above, comprise one or more ‘pharmaceutically acceptable carriers’, which include any carrier that does not itself induce the production of antibodies harmful to the individual receiving the composition. Suitable carriers are typically large, slowly metabolised macromolecules such as proteins, polysaccharides, polylactic acids, polyglycolic acids, polymeric amino acids, amino acid copolymers, trehalose (WO00/56365) and lipid aggregates (such as oil droplets or liposomes). Such carriers are well known to those of ordinary skill in the art. The vaccines may also contain diluents, such as water, saline, glycerol, etc. Additionally, auxiliary substances, such as wetting or emulsifying agents, pH buffering substances, and the like, may be present. A thorough discussion of pharmaceutically acceptable excipients is available in Remington's Pharmaceutical Sciences.

Immunogenic compositions used as vaccines comprise an immunologically effective amount of antigen, as well as any other of the above-mentioned components, as needed. By ‘immunologically effective amount’, it is meant that the administration of that amount to an individual, either in a single dose or as part of a series, is effective for treatment or prevention. This amount varies depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other rel-evant factors. It is expected that the amount will fall in a relatively broad range that can be determined through routine trials. Dosage treatment may be a single dose schedule or a multiple dose schedule (e.g. including booster doses). The vaccine may be administered in conjunction with other immunoregulatory agents.

The vaccine may be administered in conjunction with other immunoregulatory agents.

The composition may include other adjuvants in addition to (or in place of) the aluminium salt. Preferred adjuvants to enhance effectiveness of the composition include, but are not limited to: (1) oil-in-water emulsion formulations (with or without other specific immunostimulating agents such as muramyl peptides (see below) or bacterial cell wall components), such as for example (a) MF59™ (WO90/14837; Chapter 10 in ref. 27), containing 5% Squalene, 0.5% Tween 80, and 0.5% Span 85 (optionally containing MTP-PE) formulated into submicron particles using a microfluidizer, (b) SAF, containing 10% Squalane, 0.4% Tween 80, 5% pluronic-blocked polymer L121, and thr-MDP either microfluidized into a submicron emulsion or vortexed to generate a larger particle size emulsion, and (c) Ribi™ adjuvant system (RAS), (Ribi Immunochem, Hamilton, Mont.) containing 2% Squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™); (2) saponin adjuvants, such as QS21 or Stimulon™ (Cambridge Bioscience, Worcester, Mass.) may be used or particles generated therefrom such as ISCOMs (immunostimulating complexes), which ISCOMS may be devoid of additional detergent e.g. WO00/07621; (3) Complete Freund's Adjuvant (CFA) and Incomplete Freund's Adjuvant (IFA); (4) cytokines, such as interleukins (e.g. IL-1, IL-2, IL-4, IL-5, IL-6, IL-7, IL-12 (WO99/44636), etc.), interferons (e.g. gamma interferon), macrophage colony stimulating factor (M-CSF), tumor necrosis factor (TNF), etc.; (5) monophosphoryl lipid A (MPL) or 3-O-deacylated MPL (3dMPL) e.g. GB-2220221, EP-A-0689454; (6) combinations of 3dMPL with, for example, QS21 and/or oil-in-water emulsions e.g. EP-A-0835318, EP-A-0735898, EP-A-0761231; (7) oligonucleotides comprising CpG motifs [Krieg Vaccine 2000, 19, 618-622; Krieg Curr opin Mol Ther 2001 3:15-24; Roman et al., Nat. Med., 1997, 3, 849-854; Weiner et al., PNAS USA, 1997, 94, 10833-10837; Davis et al., J. Immunol., 1998, 160, 870-876; Chu et al., J. Exp. Med., 1997, 186, 1623-1631; Lipford et al., Eur. J. Immunol., 1997, 27, 2340-2344; Moldoveanu et al., Vaccine, 1988, 16, 1216-1224, Krieg et al., Nature, 1995, 374, 546-549; Klinman et al., PNAS USA, 1996, 93, 2879-2883; Ballas et al., J. Immunol., 1996, 157, 1840-1845; Cowdery et al., J. Immunol., 1996, 156, 4570-4575; Halpern et al., Cell. Immunol., 1996, 167, 72-78; Yamamoto et al., Jpn. J. Cancer Res., 1988, 79, 866-873; Stacey et al., J. Immunol., 1996, 157, 2116-2122; Messina et al., J. Immunol., 1991, 147, 1759-1764; Yi et al., J. Immunol., 1996, 157, 4918-4925; Yi et al., J. Immunol., 1996, 157, 5394-5402; Yi et al., J. immunol., 1998, 160, 4755-4761; and Yi et al., J. Immunol., 1998, 160, 5898-5906; International patent applications WO96/02555, WO98/16247, WO98/18810, WO98/40100, WO98/55495, WO98/37919 and WO98/52581] i.e. containing at least one CG dinucleotide, with 5-methylcytosine optionally being used in place of cytosine; (8) a polyoxyethylene ether or a polyoxyethylene ester e.g. WO99/52549; (9) a polyoxyethylene sorbitan ester surfactant in combination with an octoxynol (e.g. WO01/21207) or a polyoxyethylene alkyl ether or ester surfactant in combination with at least one additional non-ionic surfactant such as an octoxynol (e.g. WO01/21152); (10) an immunostimulatory oligonucleotide (e.g. a CpG oligonucleotide) and a saponin e.g. WO00/62800; (11) an immunostimulant and a particle of metal salt e.g. WO00/23105; (12) a saponin and an oil-in-water emulsion e.g. WO99/11241; (13) a saponin (e.g. QS21)+3dMPL+IL-12 (optionally +a sterol) e.g. WO98/57659; (14) other substances that act as immunostimulating agents to enhance the efficacy of the composition. Muramyl peptides include N-acetyl-muramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-2-(1′-2′-dipalmitoyl-sn-glycero-3-hydroxyphosphoryloxy)-ethylamine MTP-PE), etc.

### Further Antigens

Further antigens which can be included in the composition of the invention include:
further antigens from H. pylori such as HopX [e.g. 34], HopY [e.g. 34] and/or urease.
a protein antigen from N. meningitidis serogroup B, such as those in refs. 35 to 41, with protein '287′ (see below) and derivatives (e.g. ‘ΔG287’) being particularly preferred.
an outer-membrane vesicle (OMV) preparation from N. meningitidis serogroup B, such as those disclosed in refs. 42, 43, 44, 45 etc.
a saccharide antigen from N. meningitidis serogroup A, C, W135 and/or Y, such as the oligosaccharide disclosed in ref. 46 from serogroup C [see also ref. 47].
a saccharide antigen from Streptococcus pneumoniae [e.g. 48, 49, 50].
an antigen from hepatitis A virus, such as inactivated virus [e.g. 51, 52].
an antigen from hepatitis B virus, such as the surface and/or core antigens [e.g. 52, 53].
an antigen from hepatitis C virus [e.g. 54].
an antigen from Bordetella pertussis, such as pertussis holotoxin (PT) and filamentous haemagglutinin (FHA) from B. pertussis, optionally also in combination with pertactin and/or agglutinogens 2 and 3 [e.g. refs. 55 & 56].
a diphtheria antigen, such as a diphtheria toxoid [e.g. chapter 3 of ref. 57] e.g. the CRM₁₉₇ mutant [e.g. 58].
a tetanus antigen, such as a tetanus toxoid [e.g. chapter 4 of ref. 57].
a saccharide antigen from Haemophilus influenzae B [e.g. 47].
an antigen from N. gonorrhoeae [e.g. 35, 36, 37].
an antigen from Chlamydia pneumoniae [e.g. 59, 60, 61, 62, 63, 64, 65].
an antigen from Chlamydia trachomatis [e.g. 66].
an antigen from Porphyromonas gingivalis [e.g. 67].
polio antigen(s) [e.g. 68, 69] such as IPV or OPV.
rabies antigen(s) [e.g. 70] such as lyophilised inactivated virus [e.g. 71, RabAvert™].
measles, mumps and/or rubella antigens [e.g. chapters 9, 10 & 11 of ref. 57].
influenza antigen(s) [e.g. chapter 19 of ref. 57], such as the haemagglutinin and/or neuraminidase surface proteins.
an antigen from Moraxella catarrhalis [e.g. 72].
an antigen from Streptococcus agalactiae (group B streptococcus) [e.g. 73, 74].
an antigen from Streptococcus pyogenes (group A streptococcus) [e.g. 74, 75, 76].
an antigen from Staphylococcus aureus [e.g. 77].

The composition may comprise one or more of these further antigens.

Where a saccharide or carbohydrate antigen is used, it is preferably conjugated to a carrier protein in order to enhance immunogenicity [e.g. refs. 78 to 87]. Preferred carrier proteins are bacterial toxins or toxoids, such as diphtheria or tetanus toxoids. The CRM₁₉₇ diphtheria toxoid is particularly preferred. Other suitable carrier proteins include the N. meningitidis outer membrane protein [e.g. ref. 88], synthetic peptides [e.g. 89, 90], heat shock proteins [e.g. 91], pertussis proteins [e.g. 92, 93], protein D from H. influenzae [e.g. 94], toxin A or B from C. difficile [e.g. 95], etc. Where a mixture comprises capsular saccharides from both serogroups A and C, it is preferred that the ratio (w/w) of MenA saccharide:MenC saccharide is greater than 1 (e.g. 2:1, 3:1, 4:1, 5:1, 10:1 or higher). Saccharides from different serogroups of N. meningitidis may be conjugated to the same or different carrier proteins.

Any suitable conjugation reaction can be used, with any suitable linker where necessary.

Toxic protein antigens may be detoxified where necessary (e.g. detoxification of pertussis toxin by chemical and/or genetic means [56]).

Where a diphtheria antigen is included in the composition it is preferred also to include tetanus antigen and pertussis antigens. Similarly, where a tetanus antigen is included it is preferred also to include diphtheria and pertussis antigens. Similarly, where a pertussis antigen is included it is preferred also to include diphtheria and tetanus antigens.

Antigens are preferably adsorbed to an aluminium salt.

Antigens in the composition will typically be present at a concentration of at least 1 μg/ml each. In general, the concentration of any given antigen will be sufficient to elicit an immune response against that antigen.

Where urea is included in the composition of the invention, it is preferred not to include active urease as an antigen.

As an alternative to using proteins antigens in the composition of the invention, nucleic acid encoding the antigen may be used [e.g. refs. 96 to 104]. Protein components of the compositions of the invention may thus be replaced by nucleic acid (preferably DNA e.g. in the form of a plasmid) that encodes the protein.

### Further Anti-Helicobacter Agents

Compositions of the invention may be administered in conjunction with an antisecretory agent and/or an antibiotic effective against Helicobacter pylori. These components offer rapid relief from any existing H. pylori infection, thereby complementing the longer timescale of immunotherapy.

These may be administered in the same composition as the protein antigens, but will typically be administered separately. They may be administered at the same time as the protein antigens, but they will generally follow a separate administration protocol e.g. daily. They may be administered by the same route as the protein antigens, but they will generally be administered orally. They may be administered over the same timescale as the protein antigens, but they will generally be administered from shortly before (e.g. up to 5 to 14 days before) the first dose of protein antigen up to shortly after (e.g. up to 5 to 14 days after) the last dose of protein antigen.

Preferred antisecretory agents are proton pump inhibitors (PPIs), H2 receptor antagonists, bismuth salts and prostaglandin analogs.

Preferred PPIs are omeprazole (including S- and B- forms, Na and Mg salts etc. [e.g. 105,106]), lansoprazole, pantoprazole, esomeprazole, rabeprazole, the heterocyclic compounds disclosed in reference 107, the imidazo pyridine derivatives of reference 108, the fused dihydropyrans of reference 109, the pyrrolidine derivatives of reference 110, the benzamide derivatives of reference 111, the alkylenediamine derivatives of reference 112 etc.

Preferred H2-receptor antagonists are ranitidine, cimetidine, famotidine, nizatidine and roxatidine.

Preferred bismuth salts are the subsalicylate and the subcitrate, and also bismuth salts of antibiotics of the moenomycin group [113].

Preferred prostaglandin analogs are misoprostil and enprostil.

Preferred antibiotics are tetracycline, metronidazole, clarithromycin and amoxycillin.

Other suitable anti-H. pylori agents are disclosed in, for instance, reference 114.

### DEFINITIONS

The term “comprising” means “including” as well as “consisting” e.g. a composition “comprising” X may consist exclusively of X or may include something additional e.g. X+Y.

The term “about” in relation to a numerical value x means, for example, x±10%.

### BRIEF DESCRIPTION OF DRAWINGS

FIG. 1 shows the efficacy of prophylactic oral immunisation with H. pylori antigens [9,10] using LTK63 as adjuvant. Protection is assessed as the absence of colonies after plating of stomachs from mice which received the indicated treatments. Data are from different experiments.

FIG. 2 shows the protection of beagle conventional dogs against H. pylori infection following immunisation with whole-cell lysates by different routes. FIG. 2A shows immunogenicity in the dogs (the four bars in each graph are, from left to right: control, intragastric, intranasal, intramuscular). FIG. 2B summarises protection results. Protection was assessed as the absence of detectable bacteria by: rapid urea text, histology, immunohistochemistry, and gastric macroscopic & microscopic studies.

FIG. 3 shows the immunogenicity (3A; average titres per group) and protection conferred (3B) by intramuscular immunisation with purified VacA, CagA or NAP antigens or with whole cell lysate. Protection was assessed as described for FIG. 2.

FIG. 4 shows the immunogenicity of a mixture of CagA, VacA and NAP in beagles. Animals were immunised with either 10 μg (squares) or 50 μg (circles) of each antigen, adjuvanted with alum. The arrows show the dates of immunisation.

FIG. 5 shows the gastric biopsy results from a tolerance study in beagles.

FIGS. 6 to 13 show safety data for human administration over days 1 to 6: (6) erythema; (7) induration; (8) malaise; (9) myalgia; (10) headache; (II) arthralgia; (12) fatigue; (13) fever. Mild reactions (transient to mild discomfort) are shown as empty bars; moderate reactions (no limitation in normal daily activity) are shown as grey bars; severe reactions (unable to perform normal daily activity) are shown as black bars. The horizontal axis shows percentages.

FIGS. 14 to 19 show immunogenicity data for human administration. FIGS. 14 & 15 show antibody responses (serum IgG antibody GMT) in the monthly (14) and weekly (15) groups. FIGS. 16 & 17 show the percentage of subjects in the monthly (16) and weekly (17) groups with antibodies against all three antigens in the composition. FIGS. 18 & 19 show the cellular proliferative response to the three antigens in the monthly (18) and weekly (19) groups. In all cases the horizontal shows the number of months after the first immunisation.

### MODES FOR CARRYING OUT THE INVENTION

### HP3 Composition

Three compositions were produced for stability studies:

| Composition | |
|---|---|
| name | Components (0.5 ml dose) |
| ‘HP3’ | 25 μg/dose of each antigen (VacA, NAP, CagA); |
| | 3.75 mg/dose urea; aluminium hydroxide adjuvant |
| | 0.5 mg/dose in isotonic sodium phosphate buffer; |
| | 0.5% phenoxyethanol |
| ‘HP3 | 3.75 mg/dose urea; aluminium hydroxide adjuvant 0.5 |
| placebo’ | mg/dose in isotonic sodium phosphate buffer; |
| | 0.5% phenoxyethanol |
| ‘HP3 alum | Aluminium hydroxide adjuvant 0.5 mg/dose; NaCl 4.25 |
| control’ | mg/dose; 10 mM phosphate buffer; 0.5% phenoxyethanol |

### Stability

The stability of HP3 lots was monitored for up to 3 months at both 4° C. and 37° C.

Physico-chemical stability was assessed by measuring pH. There was no significant change in pH over the time period tested at either 4° C. or at 37° C.

Physico-chemical stability was also assessed by assaying the antigens by Western blot. There was no significant change in antigenic identity over the time period tested at either 4° C. or at 37° C.

Immunological stability was assessed by using the stored vaccines in immunisations. Groups of mice were immunised once intraperitoneally, serum samples were taken at day 28 and tested by ELISA for titration of VacA-, CagA-, and NAP-specific antibodies. The data obtained indicate that the immunogenicity of the three antigens is satisfactory for up to 3 months at 4° C. After 5 weeks of storage at 37° C., the immunogenicity of CagA was the same as for the composition stored at 4° C., whereas VacA and NAP immunogenicity was slightly reduced (but still effective).

On the basis of the results obtained under stress conditions (37° C.), the HP3 composition can be regarded as stable.

### Experimental Studies—Immunogenicity

HP3 was administered to rabbits either as a single intramuscular dose or as six doses administered once per week for six weeks. Rabbits had consistently detectable low IgG titres to all three antigens 15 days after a single immunisation. Progressively higher levels of IgG were detected in the multiple dose study starting on day 15. Levels increased by day 29 and persisted through necropsy and recovery (days 38 and 50, respectively). Untreated control animals did not mount an antibody response.

A similar study was performed in mice, and HP3 was again found to be consistently immunogenic at all doses tested (25 μg or less of each antigen per dose) following a single immunisation.

### Experimental Studies—Prophylactic Efficacy

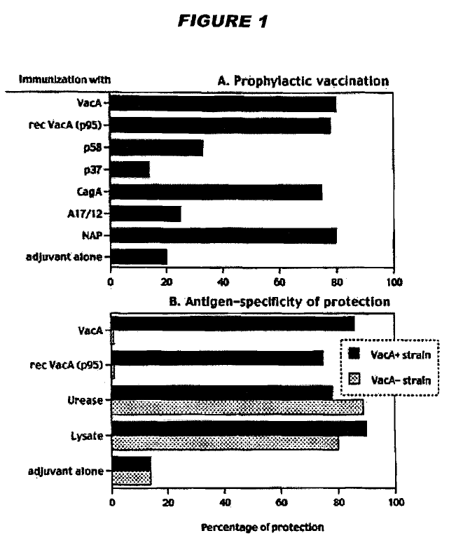

Oral immunisation of mice with recombinant or native HP antigens (VacA, CagA, NAP, and others) together with mucosal adjuvants conferred protection against subsequent challenge with H. pylori that had been freshly isolated from patients with peptic ulcer disease [FIG. 1 herein; references 9 & 10]. The immunologic mechanism underlying the observed prophylactic efficacy appears to involve MHC class II-restricted CD4⁺ cell responses, but not B cell responses [115].

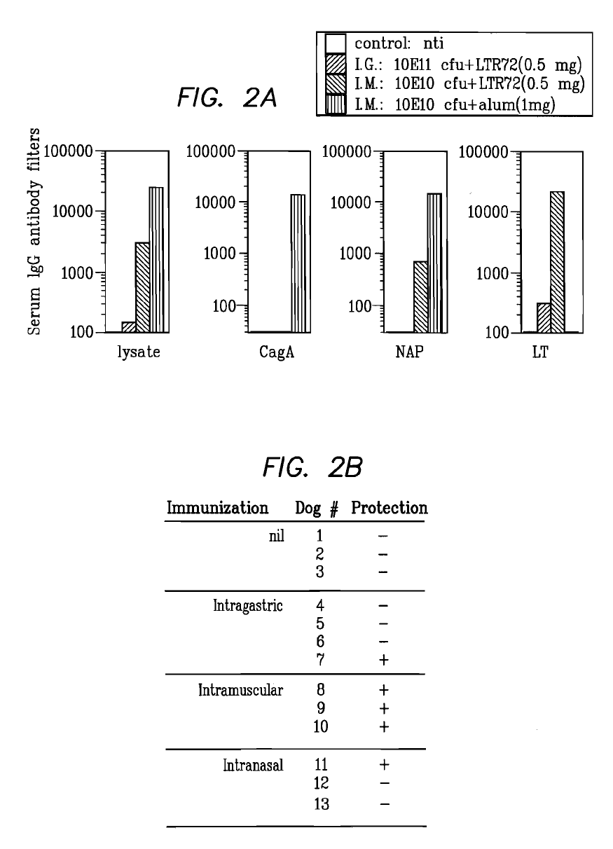

Unlike mice (which remain asymptomatic following HP infection), beagle dogs develop symptomatic infection with HP and can therefore be assessed both clinically and histologically following infection [28,116]. Using this dog model, it was determined that immunogenicity of whole-cell lysates (with aluminium hydroxide adjuvant) was greater when the lysates were administered intramuscularly compared to intranasal and intragastric administration. This intramuscular immunization also conferred protection against challenge with H. pylori (FIG. 2).

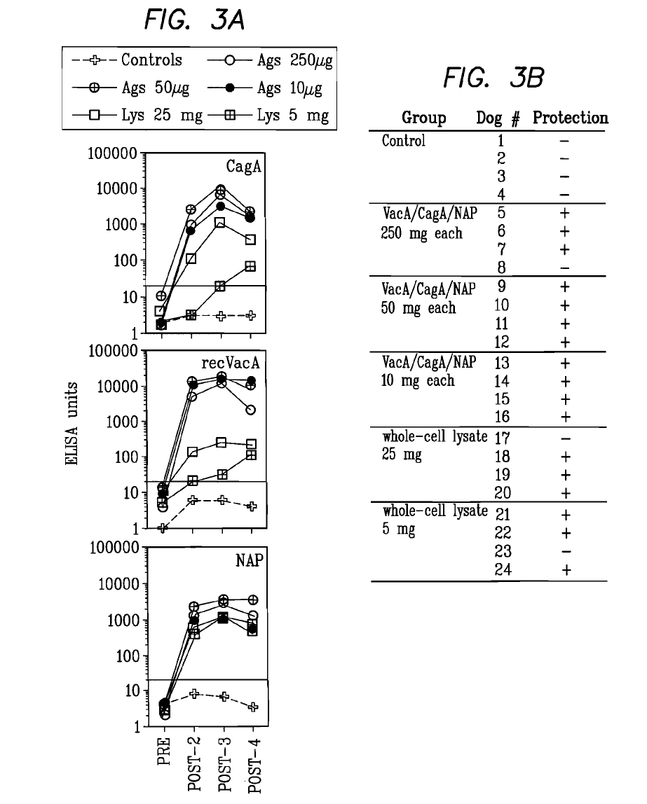

Intramuscular injection of VacA, CagA, and NAP antigens (10, 50 or 250 μg/dose of each antigen, with aluminium hydroxide adjuvant was similarly immunogenic, and conferred protection from subsequent infection (FIG. 3). In these experiments, there were no histologic or immunohistologic signs of infection in any (0/8) of the animals receiving 10 μg or 50 μg of each antigen.

### Experimental Studies—Therapeutic Efficacy

Chronic H. pylori infection was eradicated in mice given intragastric recombinant VacA and CagA together with mucosal adjuvants [117]. There was no recurrence of infection for at least three months and the mice were subsequently resistant to infection with later challenge with HP. This suggests that vaccination with these recombinant antigens induced specific immunological memory in addition to causing eradication of established infection.

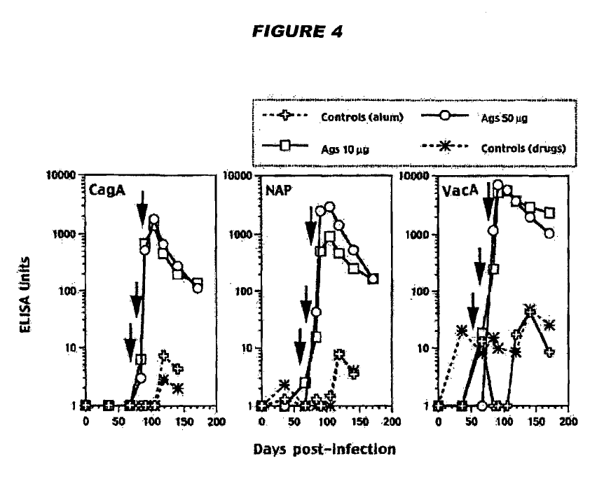

Beagle dogs infected with HP and then immunized with 10 or 50 μg of a combination of VacA+CagA+NAP (with aluminium hydroxide adjuvant) mounted a dose-dependent antigen-specific antibody response (FIG. 4). They did not show eradication of infection by mucosal urease testing at 7 and 11 weeks following immunisation. At 17 weeks, however, 2 of 4 animals treated with either dosage had negative mucosal urease tests, whereas the tests in all 4 control animals remained strongly positive. Additionally, gastric inflammatory scores showed reduced inflammation in the antigen-treated animals and no change in inflammation in the controls receiving adjuvant only.

In other experiments, beagle dogs were infected with HP and then treated intramuscularly with 10, 50, and 250 μg of antigens or bacterial lysate.

Experimental Studies—Therapeutic Efficacy in Combination with Proton Pump Inhibitor

Fourteen beagle dogs were experimentally infected with H. pylori SPM326 by intragastric administration. Three control dogs received the same treatment, but with saline substituted for bacteria.

These seventeen dogs were divided into the following experimental groups:

| Group | n | Immunisation | PPI |
|---|---|---|---|
| # 1 | 4 | HP3 | omeprazole |
| # 2 | 4 | HP3 | none |
| # 3 | 3 | HP3 alum control | omeprazole |
| # 4 | 3 | HP3 placebo | none |
| control | 3 | none | none |

Immunisations were given intramuscularly three times, at monthly intervals. Omeprazole was administered orally, daily, starting two days before the first dose of vaccine, ending two weeks after the last dose of vaccine.

No adverse clinical signs, nor body weight or temperature variations, were observed through the experimental period.

Efficacy was assessed by immunohistochemistry and histopathology on bioptic samples.

Preliminary results were obtained with biopsies taken 3 weeks after the administration of the last dose of vaccine.

In both immunised groups (#1 and #2), 3 out of 4 dogs became Helicobacter pylori-negative by immunohistochemistry, and their inflammation score was reduced compared with that observed in the pre-vaccination biopsies. No significant differences were found between the two groups.

Conversely, in both infected, control groups (#3 and #4), 3 out of 3 dogs remained Helicobacter pylori-positive by immunohistochemistry, and their inflammation score was higher than that of vaccinated groups.

### Preclinical Studies—Toxicology

Four toxicology studies were conducted to support the administration of up to 6 doses of HP3 as frequently as once per week. The third and fourth studies were designed to conform to good laboratory practice (GLP). In the GLP studies, local (injection site) and systemic toxicity were evaluated on the basis of clinical signs, physical examinations, dermal scoring, body weights and temperatures, food consumption, opthalmoscopy, clinical pathology (serum chemistry, hematology, coagulation including fibrinogen), and full macroscopic postmortem and histopathological examinations.

In addition to the toxicology studies, pertinent safety information can also be drawn from efficacy studies conducted in two other species. Immunogenicity and challenge studies were performed in mice and beagle dogs with HP3 antigens. In mice, there were no deaths attributed to HP3 formulations nor any apparent toxicity based on clinical signs. In dogs, there were no HP3 treatment-related deaths, clinical signs, changes in body weights, or clinical pathology findings.

### Irritation Study

A single dose intramuscular irritation study (code 3391.24) was performed in male NZW Rabbits. The objective of this study was to evaluate the potential for local irritant effects of the three antigens, alum and formulation excipients, including urea, in rabbits. On Day 1, twelve rabbits received three 0.5 ml intramuscular injections to the paravertebral muscle of the test and control articles as follows:

| Group | N | Site 1 | Site 2 | Site 3 |
|---|---|---|---|---|
| 1 | 6 males | HP3 | HP3 placebo | Saline |
| 2 | 6 males | HP3, but with | HP3 alum control | Saline |
| | | 7.5 mg/dose urea | | |

Clinical signs, body weights, dermal irritation, hematology, coagulation, and serum chemistry were evaluated. Three animals per group were necropsied on days 3 and 15. A macroscopic postmortem examination was conducted and injection sites, stomach, duodenum and macroscopic lesions were examined for histopathology.

There were no deaths or treatment-related effects on body weight, hematology, coagulation, or serum chemistry. Very slight erythema was seen in two animals given HP3 (Group 2, site 1). Well-defined erythema was seen in one animal given the alum control (Group 2, site 2), which diminished and was resolved completely by Day 5. There were no dermal observations in any other animals. Apparent bruising at the test sites correlated with erythema in two animals.

Injection site histopathology in animals necropsied on day 3 consisted of acute inflammation/focal necrosis attributed to needle trauma. In animals euthanized on day 15, the injection site lesions consisted of small focal clusters or accumulations of macrophages. These were typical sequelae following acute inflammation and focal necrosis seen two weeks prior. No differences in the size or character of the inflammatory components between groups or injection sites could be detected on histologic examination.

Conclusion: Under the conditions of the study, H. pylori antigens (HP3) adjuvanted with alum and containing low (3.75 mg/dose) or high (7.5 mg/dose) urea were well tolerated when administered to rabbits as a single intramuscular injection. Findings in skin (erythema) and muscle (bruising/inflammation/necrosis) were comparable across groups and sites. Local reactogenicity of formulations with or without HP3 antigens was of a low order of magnitude and was similar to either alum in saline or the HP3 placebo formulation (no antigens).

### Tolerance Study

A tolerance study (code 7795) was performed in beagle dogs infected with H. pylori. Dogs were infected with H. pylori using three oral administrations (10⁹ cfu each) administered every other day [117]. Following infection, 2 animals/sex/group were given intramuscular injections of either CagA+VacA+NAP (10 μg or 50 μg of each antigen per dose) or the alum control. A fourth group was treated with a conventional regimen including antibiotics and a proton pump inhibitor (clarithromycin 250 mg, metronidazole 250 mg, bismuth citrate 60 mg, omeprazole 20 mg). Serological and endoscopic evaluations were performed 7, 11, 17, and 27 weeks following the first administration:

| | Number of Animals | | | Route of | Treatment |
|---|---|---|---|---|---|
| Group | Males | Females | Treatment | Administr'n | Days |
| 1 | 2 | 2 | 1.0 mg alum | Intramuscular | 1, 8, 15 |
| 2 | 2 | 2 | 50 μg each | Intramuscular | 1, 8, 15 |
| | | | antigen | | |
| 3 | 2 | 2 | 10 μg each | Intramuscular | 1, 8, 15 |
| | | | antigen | | |
| 4 | 2 | 2 | Antibiotics + | b.i.d. oral | daily 1-15 |
| | | | PPI | | |

Animals in groups 2 and 3 exhibited an antibody response against each of the three antigens. A dose-response was most pronounced for the NAP component (FIG. 4). Vaccination with either antigen dose did not cause any adverse effects in terms of clinical signs, body weight, injection site reactions, body temperature, hematology, or serum chemistry as compared to the control group.

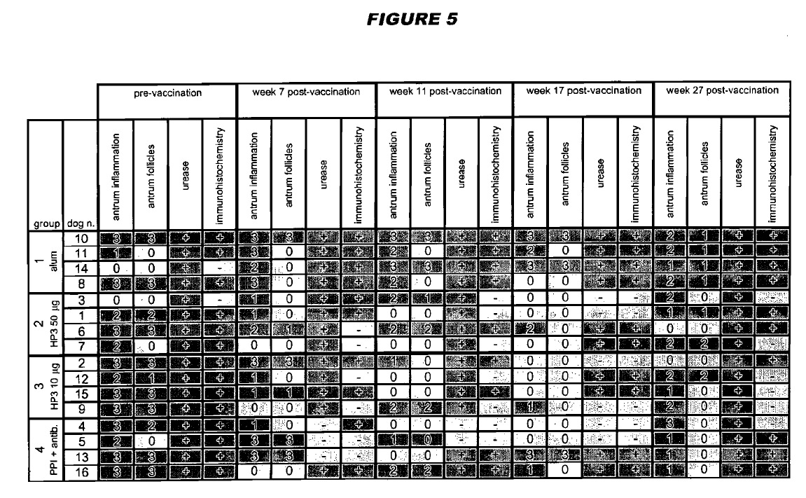

Evaluation of gastric biopsies by rapid urea test at 7 and 11 weeks post-vaccination revealed persistent H. pylori infection in all animals given adjuvant or antigen. In animals given conventional antibiotic treatment, 1/4 and 2/4 were positive for infection at weeks 7 and 11, respectively. Evaluation of gastric biopsies by immunohistochemistry using an anti-VacA-specific monoclonal antibody confirmed infection in all control animals at both timepoints. In treated groups immunohistochemistry results were variable, with 2 or 3 animals in each group scored as negative. Results are summarised in FIG. 5.

At 17 weeks, H. pylori infection was detected by rapid urease test in 4/4 in group 1, 2/4 in group 2, 2/4 in group 3, and 2/4 in group 4. In contrast to the week 7 and 11 assessments, the immunohistochemical studies confirmed the rapid urea test results.

Conclusion: The results of these studies suggest that a mixture of VacA, CagA and NAP given intramuscularly induces partial eradication of H. pylori infection and has a beneficial effect on the histological severity of post-infection gastritis. In addition, there was no evidence that the enhanced immune response elicited by the antigens was associated with any gastrontestinal or systemic adverse effects.

### GLP Safety and Tolerance Study (Single Dose)

A single dose safety and tolerability study (code UBAW-154) was performed in rabbits. The objective of this study was to evaluate the safety and tolerability of a single dose of HP3 administered intramuscularly to NZW rabbits. A secondary immunogenicity assessment was also included as a study parameter. The study consisted of three groups of 4/sex/group. Each animal either received an alum/saline mixture (Group 1), an alum/HP3 placebo formulation (Group 2), or the HP3 (Group 3). A single intramuscular dose (0.5 mL) was injected into the left quadriceps muscle on day 1 of the study. Two animals/sex/group were euthanised for a comprehensive macroscopic necropsy and tissue collection on days 3 and 15.

| | | Day 3 | Day 15 |
|---|---|---|---|
| Group | Treatment | Necropsy | Necropsy |
| 1 | Alum control | 2/sex | 2/sex |
| 2 | HP3 Placebo | 2/sex | 2/sex |
| 3 | HP3 | 2/sex | 2/sex |

Potential toxicity was evaluated based on clinical and injection site observations, body weights, physical examinations (body temperature, respiratory rate, heart rate, and capillary refill time) ophthalmic examinations, food consumption, clinical pathology (hematology, coagulation, and serum chemistry parameters), terminal organ weights, and macroscopic & microscopic evaluation of selected tissues. Serum was collected from all animals for analysis of antibody titres to HP3.

There were no deaths, no treatment-related adverse effects on any antemortem study parameters, and no relevant changes in terminal organ weights. The only dermal observation was for male number 5 (Group 2) which had a “very slight” erythema score at 24 hours post-dose that resolved by the 48-hour observation. Macroscopic postmortem findings at the injection site consisted of purple discoloration in 1/2 Group 1 females and 1/2 Group 3 males. With the exception of injection sites, there were no microscopic alterations that could be attributed to treatment. Any abnormalities noted (minor inflammatory or degenerative changes) were of the type/incidence/severity considered to be background in this strain and age of rabbit [118]. Microscopic injection site findings were minimal-to-mild and noted as follows:

| | Group Number | | | | | | | | | | | |
|---|---|---|---|---|---|---|---|---|---|---|---|---|
| | 1 (Alum) | | | | 2 (HP3 Placebo) | | | | 3 (HP3) | | | |
| | Day | | | | | | | | | | | |
| | 3 | | 15 | | 3 | | 15 | | 3 | | 15 | |
| | Number M/F | | | | | | | | | | | |
| Finding | 2 | 2 | 2 | 2 | 2 | 2 | 2 | 2 | 2 | 2 | 2 | 2 |
| Per-acute hemorrhage | 0 | 0 | 1 | 0 | 0 | 0 | 0 | 0 | 0 | 0 | 0 | 0 |
| Granulomatous inflammation | 1 | 1 | 0 | 0 | 0 | 0 | 0 | 0 | 0 | 0 | 0 | 0 |
| Acute inflammation | 0 | 0 | 0 | 0 | 0 | 0 | 0 | 0 | 1 | 0 | 0 | 0 |
| Interstitial hemorrhage | 1 | 1 | 0 | | 0 | | 0 | | 1 | | 0 | |

Based on the similarities in the histopathology regardless of treatment, the single intramuscular injection of HP3 was well tolerated by male and female rabbits. Any observations on day 3 were gone by day 15, indicating recovery or reversibility.

Analysis of day 15 serum samples for anti-NAP, CagA, and VacA antibodies indicated that low but measurable levels of IgG to all three antigens were found in all four group 3 rabbits (see above). Control rabbits were negative for antibodies.

Conclusion: Under the conditions of the study, a single 0.5 ml intramuscular injection of HP3 was well tolerated and immunogenic in male and female NZW rabbits. The local reactogenicity of HP3 was of a low order of magnitude and was similar to either the alum control or the placebo.

### GLP Safety and Tolerance Study (Multiple Dose)

A single dose safety and tolerability study (code UBAW-155) was performed in rabbits. The objective of this study was to evaluate the safety and tolerability of multiple (6) doses of HP3, once per week for six weeks by intramuscular injection to NZW rabbits. A secondary immunogenicity assessment was also included as a study parameter. The study consisted of three groups of 6/sex/group. Each animal either received the alum control, the placebo, or HP3. The dose volume was 0.5 mL alternately injected into the right and left quadriceps muscles on days 1, 8, 15, 22, 29, and 36 of the study. Three animals/sex/group were euthanised for a comprehensive macroscopic necropsy and tissue collection on days 38 and 50:

| | | | | Day 38 | | Day 50 | |
|---|---|---|---|---|---|---|---|
| | | Number | | Necropsy | | Necropsy | |
| Group | Treatment* | M | F | M | F | M | F |
| 1 | Alum/Saline | 6 | 6 | 3 | 3 | 3 | 3 |
| 2 | Alum/HP3 Placebo | 6 | 6 | 3 | 3 | 3 | 3 |
| 3 | HP3 Vaccine | 6 | 6 | 3 | 3 | 3 | 3 |

Potential toxicity was evaluated based on the following parameters: daily clinical signs, dermal injection site observations (24 and 48 hours post-dose for each dose), body weights, physical examinations (body temperature, respiratory rate, heart rate, and capillary refill time), ophthalmic examinations, food consumption, clinical pathology (hematology, coagulation, and serum chemistry parameters), terminal organ weights, full macroscopic postmortem examination, and microscopic evaluation of selected tissues:

| | | |
|---|---|---|
| Bone marrow | Injection site | Spleen |
| Eyes with optic nerve | Kidneys | Thymus |
| Femorotibial joint | Liver | Urinary bladder |
| Femur | Lung | Lesions |
| Heart | Lymph nodes | |

Observations of “very slight” dermal erythema at 24 hours post-dose were sporadic and resolved by the 48-hour observation. There were no apparent differences in the incidence or severity of dermal observations between the three groups.

There were no deaths and no treatment-related adverse effects on any antemortem study parameters (including body temperatures). There were some statistically-significant differences between groups in a few hematology, serum chemistry and coagulation parameters, however, all values were within the range of normal for this age and strain of rabbit, the changes were of small magnitude, and there was no consistent relationship to duration of dosing.

Macroscopic postmortem findings at the injection site consisted of discoloration (red/purple/tan) of the quadriceps in a few group 1 and 3 males and females. These sites of discoloration corresponded to several histologic findings, which are summarized in the following table:

| | Group Number | | | | | | | | | | | |
|---|---|---|---|---|---|---|---|---|---|---|---|---|
| | 1 (Alum) | | | | 2 (HP3 Placebo) | | | | 3 (HP3) | | | |
| | Day | | | | | | | | | | | |
| | 38 | | 50 | | 38 | | 50 | | 38 | | 50 | |
| | Number M/F | | | | | | | | | | | |
| Finding | 3 | 3 | 3 | 3 | 3 | 3 | 3 | 3 | 3 | 3 | 3 | 3 |
| Spleen | | | | | | | | | | | | |
| Follicular hyperplasia | 0 | 0 | 1 | 1 | 1 | 1 | 0 | 1 | 3 | 3 | 3 | 3 |
| Grade 1 | 0 | 0 | 1 | 1 | 1 | 1 | 0 | 1 | 1 | 1 | 3 | 2 |
| Grade 2 | 0 | 0 | 0 | 0 | 0 | 0 | 0 | 0 | 2 | 2 | 0 | 1 |
| Injection site, Right | 0 | | 0 | | 0 | | 0 | | 0 | | 0 | |
| Per-acute hemorrhage | | 1 | | 0 | | 0 | | 0 | | 2 | | 1 |
| Myofiber lysis | | 1 | | 0 | | 0 | | 0 | | 2 | | 0 |
| Eosinophil infiltration | | 0 | | 0 | | 0 | | 0 | | 0 | | 0 |
| Injection site, Left | | | | | | | | | | | | |
| Chronic inflammation | 1 | 0 | 0 | 1 | 0 | 0 | 0 | 0 | 1 | 1 | 0 | 0 |
| Interstitial hemorrhage | 1 | 0 | 0 | 0 | 0 | 0 | 0 | 0 | 0 | 1 | 0 | 0 |
| Per-acute hemorrhage | 0 | 0 | 0 | 0 | 0 | 0 | 0 | 0 | 0 | 0 | 1 | 1 |
| Myofiber lysis | 0 | 0 | 1 | 0 | 0 | 0 | 0 | 0 | 0 | 0 | 1 | 1 |
| Eosinophil infiltration | 0 | 0 | 0 | 1 | 0 | 0 | 0 | 0 | 0 | 1 | 0 | 0 |
| Proteinaceous debris | 0 | 0 | 1 | 0 | 0 | 0 | 0 | 0 | 0 | 0 | 0 | 0 |
| Granulomatous inflammation | 0 | 0 | 0 | 0 | 0 | 0 | 1 | 0 | 0 | 0 | 0 | 0 |

Two animals, one in group 1 and one in group 3, had a whitish discoloration at the injection sites noted at necropsy, but there were no correlating microscopic lesions.

Microscopic examination of the injection sites revealed that any inflammation seen in the alum controls (group 1) and HP3 placebo controls (group 2) was comparable to the HP3 vaccine injection sites. Mild granulomatous inflammation was noted in one male in group 2. The macrophage cytoplasm was distended with a granular amphophilic material, putatively alum. Granulomatous inflammation associated with i.m. administration of aluminium-based adjuvants has been reported in several species [119,120].

HP3-related microscopic alterations were noted in the spleen of all group 3 animals at both days 38 and 50. Follicular hyperplasia (B-cell dependent peri-arteriolar regions) occurred with increased incidence and severity when compared to groups 1 or 2. A slight increase in the average severity of lymphoid hyperplasia was noted for both sexes on day 38 compared to day 50. Such findings may be related to the immunological response of the rabbits to the HP3 vaccine.

With the exception of injection sites and spleen, there were no microscopic alterations that could be attributed to treatment. Any other abnormalities noted were of the type/severity/incidence considered to be background in this strain and age of rabbit [118].

Serum was collected from all animals for analysis of antibody titres to HP3. All 12 rabbits immunised with HP3 had detectable antibody titres to each of the three antigens by day 15. IgG antibody titres in all group 3 rabbits were higher on day 29 and were sustained at the same level on days 38 and 50 (See above). All control rabbits gave negative results.

Conclusion: Under the conditions of the study, administration of six 0.5 ml intramuscular injections of HP3 on a once-per-week schedule was well tolerated and immunogenic in male and female NZW rabbits. The local reactogenicity of HP3 was of a low order of magnitude and was similar to either alum in saline or the placebo formulation.

### Human Administration

A typical human immunisation will use three intramuscular injections of up to 25 μg each of NAP, CagA, and VacA antigens with alum adjuvant. The animal toxicology studies utilised a high human dose of HP3 in rabbits weighing up to approximately 4 kg. An adult body weight of 60 kg can be used as a conservative estimate. Therefore, on a body weight basis, each dose given to these rabbits would be at least 15 times higher than in a human adult. Also, the triple human regimen was exceeded by an additional three doses in the multiple-dose rabbit study.

Based on these toxicity and immunogenicity results, it can thus be expected that an immunotherapeutic (once per week for three weeks) or a prophylactic (once per month for three months) clinical regimen of intramuscular injections of 10 μg/dose or 25 μg/dose of CagA, VacA and NAP will be immunogenic and well tolerated in humans. Any local effects should be comparable to those seen with alum adjuvant and systemic effects should be consistent with other intramuscular administrations of protein antigens adjuvanted with alum.

For human use, a typical vaccine is a sterile preparation of purified CagA, VacA and NAP, with aluminium hydroxide adjuvant, in an isotonic buffer solution for intramuscular injection. The H. pylori antigens are expressed in genetically-engineered E. coli cells, utilising plasmid vector expression systems. Because of the relative insolubility of the VacA antigen, the vaccine will include urea in the amount of 2.9-4.1 mg/dose. The vaccine is provided in a pre-mixed format in syringes containing the antigens and the adjuvant. These syringes should be stored refrigerated between 2-8° C. until ready for administration. The vaccine should be shaken before use. The vaccination site should be disinfected with a skin disinfectant (e.g. 70% alcohol). Before vaccination, the skin must be dry again. The content of pre-mixed single-dose vaccine in the syringe (0.5 ml) is applied intramuscularly into alternating sides of the upper arm (M. deltoideus) using a 1 to 1½ inch needle. Two alternative vaccine compositions for human use have the following components in a single 0.5 ml dose and have a pH in the range 6.5 to 7.5:

| | Amount per final dose | | | |
|---|---|---|---|---|
| Component | Low dose | | High dose | |
| Aluminium hydroxide adjuvant | 0.5 | mg | 0.5 | mg |
| NAP | 10 | μg | 25 | μg |
| CagA | 10 | μg | 25 | μg |
| VacA | 10 | μg | 25 | μg |
| Sodium phosphate | 10 mM (0.69 mg) | | 10 mM (0.69 mg) | |
| (NaH₂PO₄•H₂O) | | | | |
| Sodium chloride (NaCl) | 2.13-2.77 | mg | 2.13-2.77 | mg |
| Urea | 2.9-4.1 | mg | 2.9-4.1 | mg |
| H₂O | Up to 0.5 | mL | Up to 0.5 | mL |

Trace amounts of chloramphenicol may also be present.

Human Testing—Safety and Immunogenicity These two compositions (and a placebo in which antigens were omitted) were tested in humans in a randomised, controlled, single-blind, dose-ranging, and schedule-optimising study with the aim of evaluating safety and immunogenicity in healthy adults. Two test populations were used: one negative for H. pylori infection (57 patients) and the other positive for H. pylori infection (56 patients). Compositions were administered as 0.5 ml doses from pre-filled syringes.

The 57 HP-negative volunteers were split into seven groups to receive the high (H; 25 μg of each antigen) or low (L; 10 μg of each antigen) dose vaccine, or the placebo (P; no antigen) with two different administration schedules. The first dose was given at time zero. In groups 1 to 5, three subsequent doses were given at 1, 2 and 4 months (‘monthly’ groups). In groups 6 & 7, two subsequent doses were given at 1 and 2 weeks (‘weekly’ groups):

| Group | n | First dose | Second dose | Third dose | Fourth dose |
|---|---|---|---|---|---|
| 1 | 7 | L | L | L | P |
| 2 | 7 | H | H | H | P |
| 3 | 7 | L | L | P | L |
| 4 | 8 | H | H | P | H |
| 5 | 9 | P | P | P | P |
| 6 | 9 | L | L | L | — |
| 7 | 10 | H | H | H | — |

Demographic data for the 57 volunteers were as follows:

| | | Monthly doses | Weekly doses | All patients |
|---|---|---|---|---|
| Parameter | | (n = 38) | (n = 19) | (n = 57) |
| Age | mean (years) | 29.9 | 28.9 | 29.6 |
| | standard devⁿ | 6.3 | 5.7 | 6.1 |
| | range | 20-40 | 20-40 | 20-40 |
| Sex | (% male) | 53 | 37 | 47 |

| | | | |
|---|---|---|---|
| Ethnicity | 100% | 100% | 100% |
| | caucasian | caucasian | caucasian |

### Safety

The following safety parameters were monitored:
Local and systemic reactions (up to day 6 post-injection).
Adverse and serious events (for entire study period).
Standard lab parameters i.e. serum chemistries and renal function (Na, K, Cl, HCO₃, urea, creatinine), complete blood count (WBC and differential, Hb, haematocrit, platelets), liver function (ALT, AST, alkaline phosphatase, bilirubin, prothrombin time, total protein, albumin).

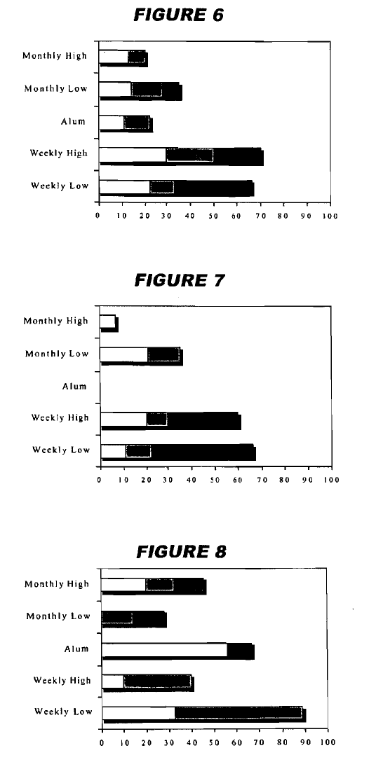

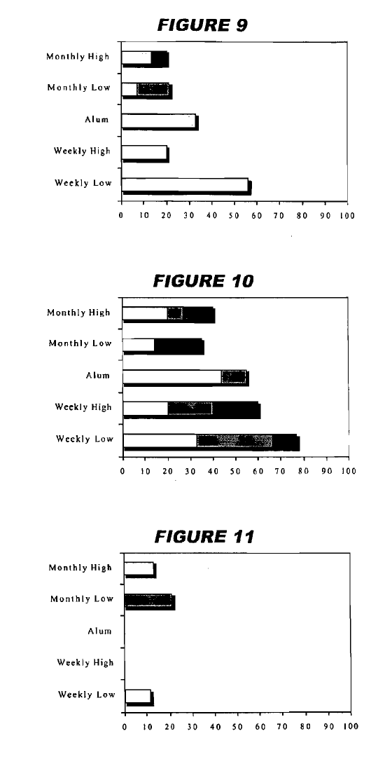

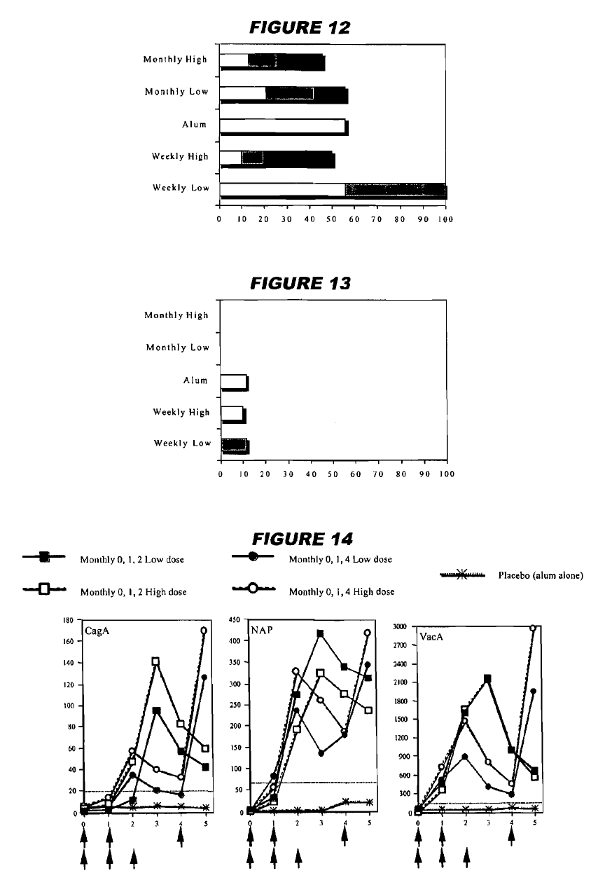

Data on erythema, induration, malaise, myalgia, headache, arthralgia, fatigue and fever are shown, in that order, in FIGS. 6 to 13. FIGS. 6 & 7 show local reactions, whereas FIGS. 8 to 13 show systemic reactions. Short-lasting pain was reported by around 89% of non-placebo subjects, compared to 78% of placebo subjects. Pain was predominantly mild and resolved after injection.

Systemic reactogenicity results are summarised in the following table:

| Adverse event | Monthly | Weekly | Placebo |
|---|---|---|---|
| (frequency ≧ 5%) | (n = 29) | (n = 19) | (n = 9) |
| Any adverse event | 14 | 15 | 7 |
| Administration site reactions | 8 | 11 | 5 |
| and general disorders | | | |
| Gastrointestinal symptoms | 3 | 3 | 2 |
| Infections | 3 | 3 | 0 |
| Musculo-skeletal symptoms | 2 | 0 | 0 |
| Nervous system disturbances* | 2 | 6 | 0 |
| Skin and subcutaneous tissue | 2 | 0 | 1 |
| manifestations | | | |

*headache, dizziness, akinesia, disturbances of alertness

The frequency and severity of local and systemic reactions were as expected in this population. Adverse events were mild in nature, transitory (lasting from a few hours up to an average two days), and were well in agreement with previous observations during clinical studies with aluminium hydroxide adjuvant. No serious adverse events related to the administration of the composition occurred in the volunteers. Local reactions were not frequent, except for local pain at the injection site in all groups. Induration and erythema occurred more often in the ‘weekly’ groups. The most frequently reported solicited systemic reactions among all groups, of any severity, were fatigue, headache and malaise. Local and systemic post-immunisation reactions were usually mild and resolved within 24-72 hours. Administration of the composition does not significantly alter laboratory parameters. Compositions of the invention are therefore safe for human administration.

### Immunogenicity

The following immunogenicity parameters were monitored:
Serum IgG specific for CagA, VacA and NAP.
Proliferative responses driven by CagA, VacA and NAP.

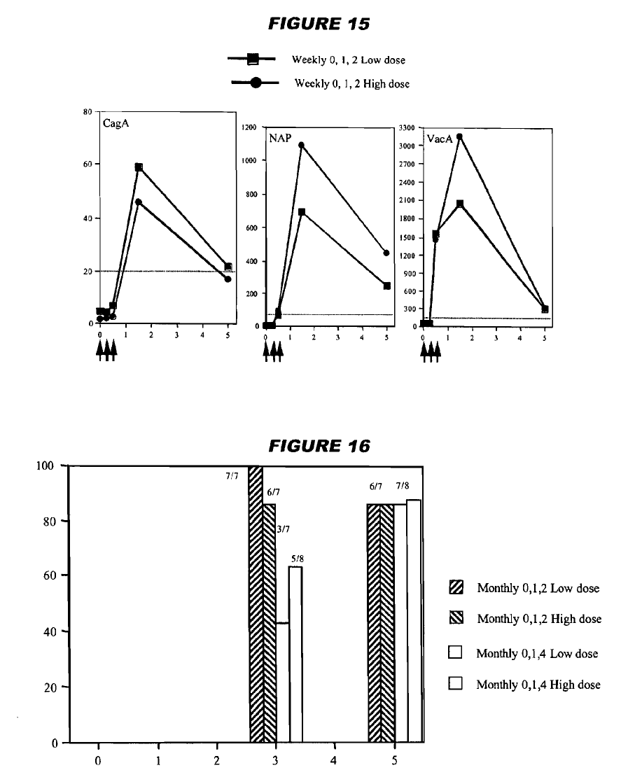

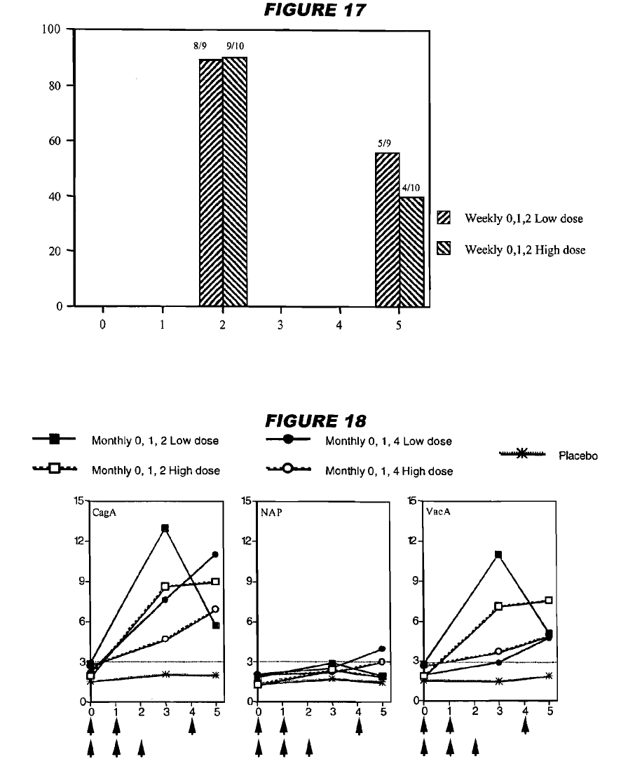

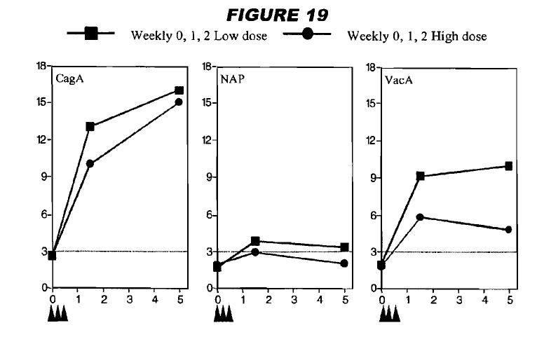

Immune responses are shown in FIG. 14 to 19. These data show that the composition is immunogenic both at antibody and cellular level in all vaccination groups. More than 85% of subjects mounted a significant antibody response to CagA, VacA and NAP after the third immunisation. The majority of subjects maintained antibody titres above the cut-off limits to all three antigens months after the 3rd dose. The majority of the subjects exhibited a significant antigen-specific cellular proliferative response (particularly CagA and VacA). The composition induces antigen-specific memory, with the antibody response being boostable and significant proliferative responses to at least two of the antigens detectable up to ≧3 months after the third immunisation

It will be understood that the invention has been described by way of example only and modifications may be made whilst remaining within the scope and spirit of the invention.

### REFERENCES

### The Contents of which are Hereby Incorporated by Reference

1—Del Giudice et al. (2001) Annu. Rev. Immunol. 19:523-563.
2—Chen et al. (2000) Exp. Opin. Ther. Patents 10:1221-1232.
3—Telford et al. (1997) Curr. Opin. Immunol. 9:498-503.
4—Dundon et al. (2001) Int. J. Med. Microbiol. 290:647-658.
5—Telford et al. (1994) TIBTECH 12:420-426.
6—Tomb et al. (1997) Nature 388:539-547.
7—Alm et al. (1999) Nature 397:176-180.
8—Marchetti et al. (1995) Science 267:1655-1658.
9—Marchetti et al. (1998) Vaccine 16:33-37.
10—Satin et al. (2000) J. Exp. Med. 191:1467-1476.
11—Covacci & Rappuoli (2000) J. Exp. Med. 19:587-592.
12—International patent application WO93/18150.
13—Covacci et al. (1993) Proc. Natl. Acad. Sci. USA 90: 5791-5795.
14—Tummuru et al. (1994) Infect. Immun. 61:1799-1809.
15—Mukhopadhyay et al. (2000) J. Bacteriol. 182: 3219-3227.
16—Telford et al. (1994) J. Exp. Med. 179:1653-1658.
17—Ji et al. (2000) Infect. Immun. 68: 3754-3757.
18—Manetti et al. (1995) Infect. Immun. 63:4476-4480.
19—Manetti et al. (1997) Infect. immun. 65:4615-4619.
20—Evans et al. (1995) Gene 153:123-127.
21—International patent applications WO96/01272 & WO96/01273, especially SEQ ID NO:6.
22—International patent application WO97/25429.
23—Tonello et al. (1999) Mol. Microbiol. 34:238-246.
24—International patent application WO99/53310.
25—Covacci et al. (1999) Science 284:1328-1333.
26—Ross et al. (1991) J Clin Pathol. 44:876-878.
27—Vaccine Design: subunit & adjuvant approach (1995) Powell & Newman (ISBN: 030644867X)
28—Rossi et al. (1999) Infect. Immun. 67:3112-3120.
29—International patent application WO98/20734.
30—Sarno et al. (2000) Pediatr. Infect. Dis. J. 19:839-842.
31-Savarino et al. (1999) Cut 45 Suppl. 1:118-122.
32—Goddard & Logan (1997) Aliment. Pharmacol. Ther. 11:641-649.
33—Vaira et al. (2000) Gastroenterol. Clin. North Am. 29:917-923.
34—International patent application WO98/04702.
35—International patent application WO99/24578.
36—International patent application WO99/36544.
37—International patent application WO99/57280.
38—International patent application WO00/22430.
39—Tettelin et al. (2000) Science 287:1809-1815.
40—International patent application WO96/29412.
41—Pizza et al. (2000) Science 287:1816-1820.
42—International patent application WO01/52885.
43—Bjune et al. (1991) Lancet 338(8775):1093-1096.
44—Fukasawa et al. (1999) Vaccine 17:2951-2958.
45—Rosenqvist et al. (1998) Dev. Biol. Stand. 92:323-333.
46—Costantino et al. (1992) Vaccine 10:691-698.
47—Costantino et al. (1999) Vaccine 17:1251-1263.
48—Watson (2000) Pediatr Infect Dis J 19:331-332.
49—Rubin (2000) Pediatr Clin North Am 47:269-285, v.
50—Jedrzejas (2001) Microbiol Mol Biol Rev 65:187-207.
51—Bell (2000) Pediatr Infect Dis J 19:1187-1188.
52—Iwarson (1995) APMIS103:321-326.
53—Gerlich et al. (1990) Vaccine 8 Suppl:S63-68 & 79-80.
54—Hsu et al. (1999) Clin Liver Dis 3:901-915.
55—Gustafsson et al. (1996) N. Engl. J. Med. 334:349-355.
56—Rappuoli et al. (1991) TIBTECH 9:232-238.
57—Vaccines (1988) eds. Plotkin & Mortimer. ISBN 0-7216-1946-0.
58—Del Guidice et al. (1998) Molecular Aspects of Medicine 19:1-70.
59—WO02/02606.
60—Kalman et al. (1999) Nature Genetics 21:385-389.
61—Read et al. (2000) Nucleic Acids Res 28:1397-406.
62—Shirai et al. (2000) J. Infect. Dis. 181 (Suppl 3):S524-S527.
63—International patent application WO99/27105.
64—International patent application WO00/27994.
65—International patent application WO00/37494.
66—International patent application WO99/28475.
67—Ross et al. (2001) Vaccine 19:4135-4142.
68—Sutter et al. (2000) Pediatr Clin North Am 47:287-308.
69—Zimmerman & Spann (1999) Am Fam Physician 59:113-118, 125-126.
70-Dreesen (1997) Vaccine 15 Suppl:S2-6.
71—MMWR Morb Mortal Wkly Rep 1998 Jan. 16; 47(1):12, 19.
72—McMichael (2000) Vaccine 19 Suppl 1:S101-107.
73—Schuchat (1999) Lancet 353(9146):51-6.
74—International patent application PCT/GB01/04789.
75—Dale (1999) Infect Dis Clin North Am 13:227-43, viii.
76—Ferretti et al. (2001) PNAS USA 98: 4658-4663.
77—Kuroda et al. (2001) Lancet 357(9264):1225-1240; see also pages 1218-1219.
78—Ramsay et al. (2001) Lancet 357(9251):195-196.
79—Lindberg (1999) Vaccine 17 Suppl 2:S28-36.
80—Buttery & Moxon (2000) J R Coll Physicians Lond 34:163-168.
81—Ahmad & Chapnick (1999) Infect Dis Clin North Am 13:113-133, vii.
82—Goldblatt (1998) J. Med. Microbiol. 47:563-567.
83—European patent 0 477 508.
84—U.S. Pat. No. 5,306,492.
85—International patent application WO98/42721.
86—Conjugate Vaccines (eds. Cruse et al.) ISBN 3805549326, particularly vol. 10:48-114.
87—Hermanson (1996) Bioconjugate Techniques ISBN: 0123423368 or 012342335X.
88—European patent application 0372501.
89—European patent application 0378881.
90—European patent application 0427347.
91—International patent application WO93/17712.
92—International patent application WO98/58668.
93—European patent application 0471177.
94—International patent application WO00/56360.
95—International patent application WO00/61761.
96—Robinson & Torres (1997) Seminars in Immunology 9:271-283.
97—Donnelly et al. (1997) Annu Rev Immunol 15:617-648.
98-Scott-Taylor & Dalgleish (2000) Expert Opin Investig Drugs 9:471-480.
99—Apostolopoulos & Plebanski (2000) Curr Opin Mol Ther 2:441-447.
100—Ian (1999) Curr Opin Mol Ther 1:116-120.
101—Dubensky et al. (2000) Mol Med 6:723-732.
102—Robinson & Pertmer (2000) Adv Virus Res 55:1-74.
103—Donnelly et al. (2000) Am J Respir Crit. Care Med 162(4 Pt 2):S190-193.
104—Davis (1999) Mt. Sinai J. Med. 66:84-90.
105—WO98/54171
106—WO99/00380
107—WO99/28322.
108—U.S. Pat. No. 6,265,415.
109—U.S. Pat. No. 6,160,119.
110—U.S. Pat. No. 5,925,667.
111—U.S. Pat. No. 5,891,890.
112—U.S. Pat. No. 6,001,880.
113—U.S. Pat. No. 6,077,830.
114—Chen et al. (2000) Exp. Opin. Ther. Patents 10:1221-1232.
115—Ermak et al. (1998) J. Exp. Med. 188:2277-2288.
116—Rossi et al. (2000) Infect. Immun. 68:4769-4772.
117—Ghiara et al. (1997) Infect. Immun. 65:4996-5002.
118—Percy Pathology of Laboratory Rodents and Rabbits. Iowa State University Press; 1993.
119—Goto et al. (1982) Microbiol. Immunol. 26(12):1121-1132.
120—Goto et al. (1997) Vaccine 15:1364-1371.

1. (canceled)

2. A composition comprising: (a) Helicobacter pylori cytotoxicity associated antigen (Ca-2A) vacuolating toxin (VacA) and neutrophil activating protein (NAP); (b) an aluminium salt adjuvant; (c) a buffer solution; and (d) urea.

3. The composition of claim 2, wherein CagA, VacA and NAP are each present at a concentration of 10 μg/dose.

4. The composition of claim 2, wherein CagA, VacA and NAP are each present at a concentration of 20 μg/ml.

5. The composition of claim 2, wherein CagA, VacA and NAP are each present at a concentration of 25 μg/dose.

6. The composition of claim 2, wherein CagA, VacA and NAP are each present at a concentration of 50 μg/ml.

7. The composition of claim 2, wherein the alum salt is an aluminium hydroxide.

8. The composition of claim 7, wherein the aluminium hydroxide has a concentration of 1 mg/ml.

9. The composition of claim 2, wherein the buffer solution is a phosphate buffer.

10. The composition of claim 2, wherein said composition is buffered to a pH of between 6 and 8.

11. The composition of claim 2, wherein the composition is isotonic.

12. The composition of claim 2, wherein the composition is sterile.

13. The composition of claim 2, adapted for intramuscular administration.

14. The composition of claim 13, adapted for administration as an injectable.

15. The composition of claim 2, wherein urea is present in an amount sufficient to ensure that VacA remains soluble.

16. The composition of claim 2, further comprising
a protein antigen from N. meningitidis;
an outer-membrane vesicle (OMV) preparation from N. meningitidis;
a saccharide antigen from N. meningitidis;
a saccharide antigen from Streptococcus pneumoniae;
an antigen from hepatitis A, B and/or C virus;
an antigen from Bordetella pertussis;
a diphtheria antigen;
a tetanus antigen;
a protein antigen from Helicobacter pylori;
a saccharide antigen from Haemophilus influenzae;
an antigen from N. gonorrhoeae;
an antigen from Chlamydia pneumoniae;
an antigen from Chlamydia trachomatis;
an antigen from Porphyromonas gingivalis;
polio antigen(s);
rabies antigen(s);
measles, mumps and/or rubella antigens;
influenza antigen(s);
an antigen from Moraxella catarrhalis;
an antigen from Streptococcus agalactiae;
an antigen from Streptococcus pyogenes; or
an antigen from Staphylococcus aureus.

17. The composition of claim 2, being an immunogenic composition.

18. The composition of claim 2, wherein said composition is a vaccine composition.

19. The composition of claim 2, further comprising an antisecretory agent and/or an antibiotic effective against Helicobacter pylori.

20. The composition of claim 19, wherein the antisecretory agent is a proton pump inhibitor, a H2 receptor antagonist, a bismuth salt or a prostaglandin analog.

21. A kit comprising a syringe, a needle, and the composition of claim 2.

22. The kit of claim 21 wherein the composition is within the syringe.

23. The kit of claim 21, further comprising an antisecretory agent and/or an antibiotic effective against Helicobacter pylori.

24. The kit of claim 23, wherein the antisecretory agent is a proton pump inhibitor, a H2 receptor antagonist, a bismuth salt or a prostaglandin analog.

25. A process for producing the composition claim 2, comprising the step of admixing H. pylori CagA, VacA and NAP proteins, an aluminium salt, a buffer solution and urea.

26. A method for inducing an immune response in a mammal against cytotoxicity associated antigen (CagA) vacuolating toxin (VacA), and neutrophil activating protein (NAP) by administering to said mammal (a) the composition of claim 2 and (b) an antisecretory agent and/or an antibiotic effective against Helicobacter pylori.

27. A method for preventing or treating an infection and/or disease caused by Helicobacter pylori in a mammal comprising administering to said mammal (a) the composition of claim 2 and (b) an antisecretory agent and/or an antibiotic effective against Helicobacter pylori.

28. A process for monitoring the efficacy of a composition of claim 2, wherein one or more of the following tests is performed on a patient to whom the composition has been administered: urease breath test, stool antigen shedding, and/or immunological analysis.

29. The process of claim 28, wherein the process monitors prophylactic efficacy.

30. The process of claim 28, wherein the process monitors therapeutic efficacy.

31. (canceled)
